(19) United States
(12) Patent Application Publication
(10) Pub. No.: US 2011/0245619 A1
(43) Pub. Date: Oct. 6, 2011

(54) SURGICAL ACCESS DEVICE

(75) Inventor: Matthew D. Holcomb, Lebanon, OH (US)

(73) Assignee: Ethicon Endo-Surgery, Inc., Cincinnati, OH (US)

(21) Appl. No.: 12/752,701

(22) Filed: Apr. 1, 2010

(51) Int. Cl. A61B 1/32

(52) U.S. Cl. 600/206; 600/201

(57) ABSTRACT

A surgical device is disclosed which can be utilized to access a surgical site within a patient. The surgical device can comprise a member insertable into a patient, a base member configured to be positioned against the patient, and a flexible sleeve extending therebetween. The surgical device can further comprise an upper member which can be assembled to the base member such that it can be rotated about an axis. The upper member and the base member can comprise corresponding retention features and slots which can permit the upper member to be rotated relative to the base member yet prevent the upper member from being disassembled from the base member unless the upper member and the base member have been aligned in a unique orientation.

### BACKGROUND

i. Field of the Invention

The present application relates to methods and devices for laparoscopic surgical procedures and, more particularly, to hand-assisted, laparoscopic procedures.

ii. Description of the Related Art

In a minimally invasive, laparoscopic surgical procedure, a surgeon may place a number of small ports into the abdomen to gain access into the abdominal cavity of the patient. A surgeon may use, for example, a port for insufflating the abdominal cavity to create space, a port for introducing a laparoscope for viewing, and a number of other ports for introducing surgical instruments for operating on tissue. The benefits of minimally invasive procedures compared to open surgery procedures for treating certain types of wounds and diseases are now well-known to include faster recovery time and less pain for the patient, better outcomes, and lower overall costs.

In traditional, open surgery, surgeons may use their hands, together with surgical instrumentation, to manipulate tissues, to perform particular steps of the procedure and to obtain tactile feedback through their fingertips to verify the nature of particular tissues. Also in open surgery, the size and shape of instruments that a surgeon may place into the abdominal cavity, as well as the size and shape of tissues that a surgeon may remove, obviously is not nearly as limited as in laparoscopic surgery.

Hand-assisted, laparoscopic surgery (“HALS”) combines some of the benefits of both the open and the laparoscopic methods. In a HALS procedure, a surgeon still places small ports into the abdomen to insufflate, to view and to introduce instruments into the abdominal cavity. In a HALS procedure, however, a surgeon also creates an incision into the abdominal wall large enough to accommodate the surgeon's hand. The incision may be retracted and draped to provide a suitably sized and protected opening. A surgeon may also place a laparoscopic access device, also referred to as a lap disc, into the incision to maintain insufflation in the abdominal cavity while the surgeon's hand is either inserted into the cavity though the device or removed from the cavity. The advent of HALS and the lap disc creates numerous opportunities for creating and/or improving surgical devices and methods.

The foregoing discussion is intended only to illustrate various aspects of the related art in the field of the invention at the time, and should not be taken as a disavowal of claim scope.

### FIGURES

Various features of the embodiments described herein are set forth with particularity in the appended claims. The various embodiments, however, both as to organization and methods of operation, together with advantages thereof, may be understood in accordance with the following description taken in conjunction with the accompanying drawings as follows.

FIG. 1 is a partially sectioned front view of an access device.

FIG. 2 is a perspective view of a first aspect of a base.

FIG. 3 is a top view of the base of FIG. 2.

FIG. 4 is a sectional side view of a first aspect of a seal assembly for use with the base of FIG. 1.

FIG. 5 is a top view of the seal assembly of FIG. 4.

FIG. 6 is a sectional side view of an embodiment of the base of FIG. 2.

FIG. 7 is a sectional side view of a laparoscopic access device including the base of FIG. 6.

FIG. 8 is an expanded cross-sectional view of an instrument support.

FIG. 9 is a view of the instrument support of FIG. 8 in an assembled state.

FIG. 10 is a partially-sectioned view of an access device.

FIG. 11 is a perspective view of an upper portion assembled to a lower ring of the access device of FIG. 10.

FIG. 12 is a side view of the upper portion and the lower ring of the access device of FIG. 10.

FIG. 13 is a cross-sectional view of the upper portion and the lower ring of the access device of FIG. 10.

FIG. 14 is another cross-sectional view of the upper portion and the lower ring of the access device of FIG. 10 which is perpendicular to the cross-section of FIG. 13.

FIG. 15 is a side view of the upper portion of the access device of FIG. 10.

FIG. 16 is a perspective view of the upper portion of the access device of FIG. 10.

FIG. 17 is a top view of the upper portion of the access device of FIG. 10.

FIG. 18 is a bottom perspective view of the lower ring of FIG. 10.

FIG. 19 is a side view of the lower ring of FIG. 10.

FIG. 20 is another side view of the ring of FIG. 10.

Corresponding reference characters indicate corresponding parts throughout the several views. The exemplifications set out herein illustrate various embodiments of the invention, in one form, and such exemplifications are not to be construed as limiting the scope of the invention in any manner.

### DESCRIPTION

Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

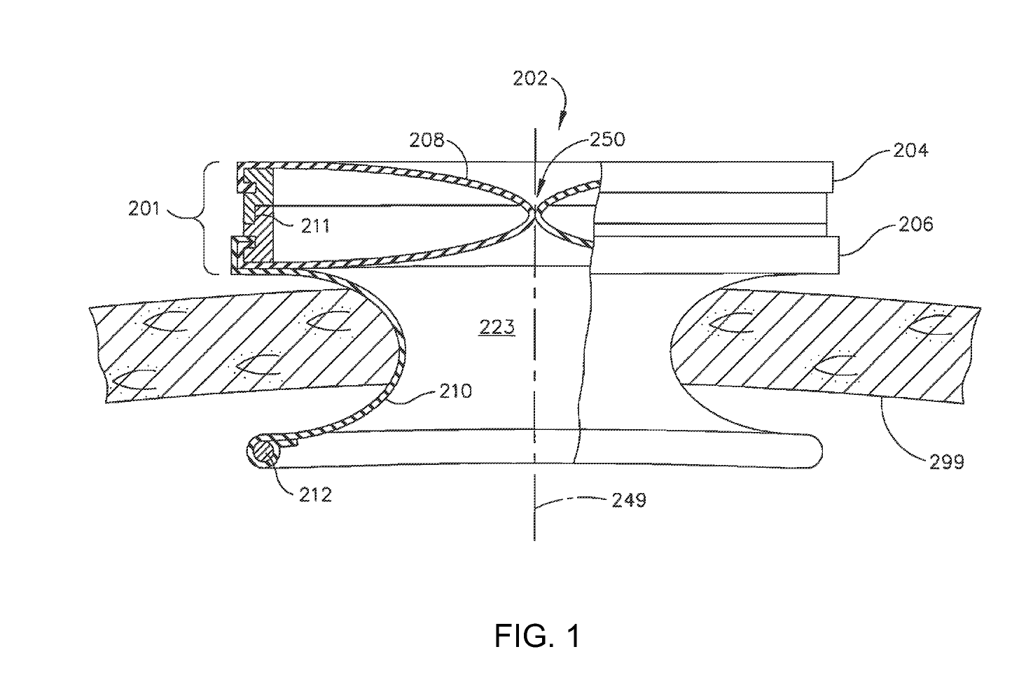

FIG. 1 is a partially sectioned front view of an access device 202 positioned in a body wall 299 of a patient. Access device 202 is disclosed in U.S. Pat. No. 6,110,154, which issued to Shimomura et al. on Aug. 29, 2000, and is entitled VALVE AND VALVED TROCAR JACKET TUBE, the entire disclosure of which is incorporated by reference herein. Access device 202 includes an upper ring 204, a lower ring 206, a first cylindrical elastic member 208 (or first elastic member 208), a second cylindrical elastic member 210 (also referred to as second elastic member 210 and sleeve 210), and a resilient member 212. First elastic member 208 and second elastic member 210 are each made of a thin-walled, silicone rubber tubing material, or any one of a number of other elastic, biocompatible materials in sheet or tube form. The ends of first elastic member 208 are assembled with upper ring 204 and lower ring 206, respectively, to form a hyperboloid (“hour glass”) shape defining an opening 250 centered on a vertical axis 249 of access device 202. Similarly, the ends of second elastic member 210 are assembled with lower ring 206 and resilient ring 212 to form a hyperboloid shape and defining a passageway 223 therethrough. The surgeon may position second elastic member 210 in the body wall 299 of the patient by pushing resilient ring 212 (while folded) through the surgical incision. Once in the body cavity, resilient ring 212 resumes an approximately circular shape to sealingly retain access device 202 in body wall 299. An annular interface 211 between upper ring 204 and lower ring 206 frictionally holds the relative angular orientation of upper ring 204 and lower ring 206 in order to maintain the size of opening 250. The frictional holding force is easily overcome by the surgeon turning either one of upper ring 204 and lower ring 206 while holding the other. Additionally, upper ring 204 and lower ring 206 may each be molded from a plastic to have interlocking features around the perimeter of their mating surfaces. The surgeon may accordingly adjust the relative angular position about vertical axis 249 of upper ring 204 with respect to lower ring 206, and thus set the size of opening 250 to numerous diameters ranging from a fully closed configuration to a fully open configuration. The surgeon may adjust opening 250, therefore, to seal against the surgeon's hand or one or more surgical instruments extending through opening 250, providing the ability to insufflate the body cavity with carbon dioxide during the surgical procedure.

Upper ring 204, lower ring 206 and first elastic member 208 are also referred to together as a valve subassembly 201. As will become apparent to those skilled in the art, the aspects and features described herein are also applicable to surgical access devices having other types of valve assemblies such as, for example, those including a hydrophilic gel material with a sealable slit opening for surgical access into the body cavity.

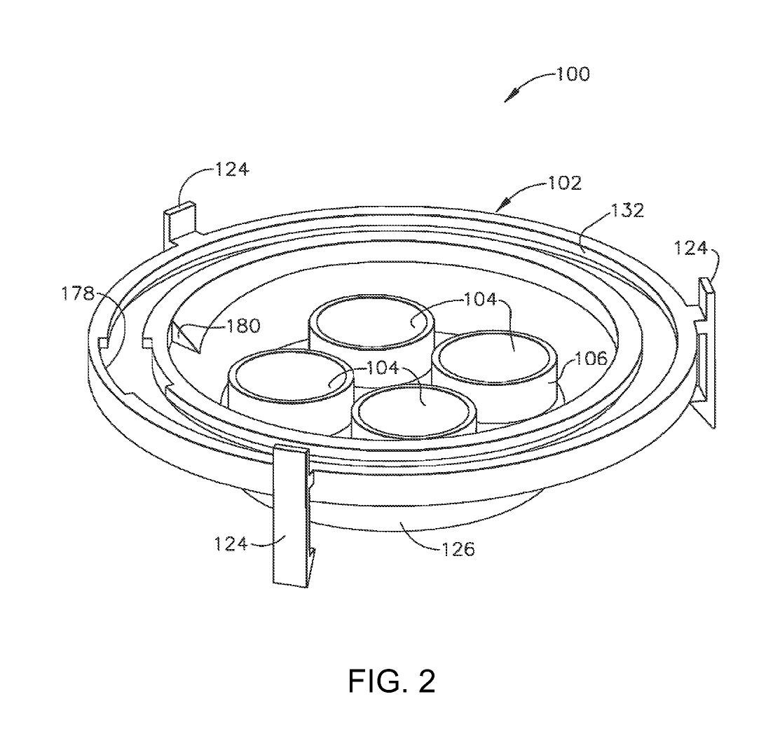

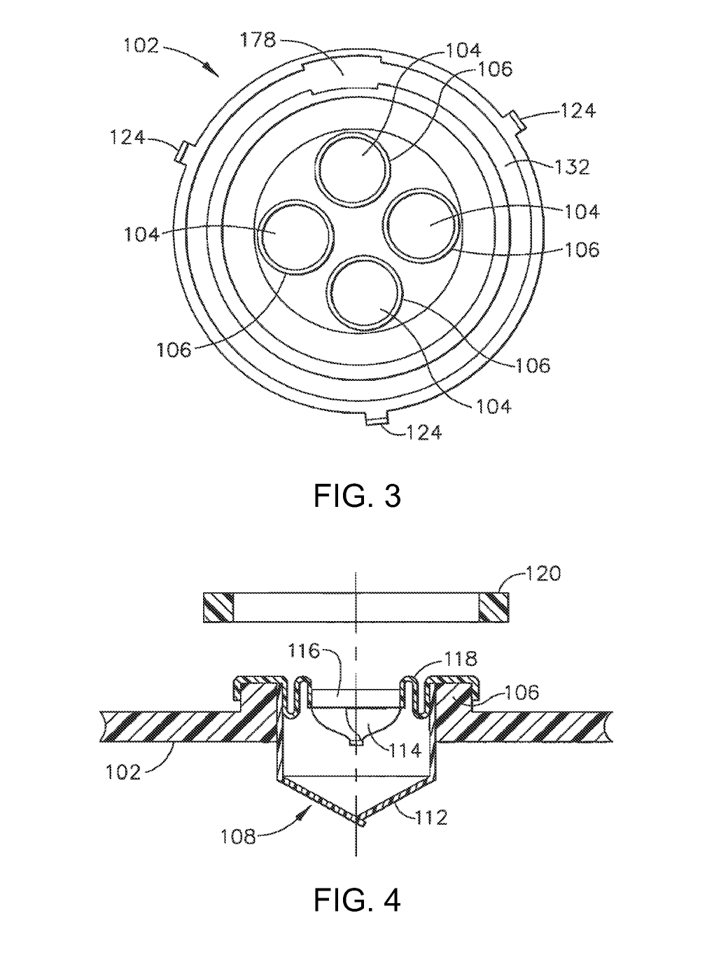

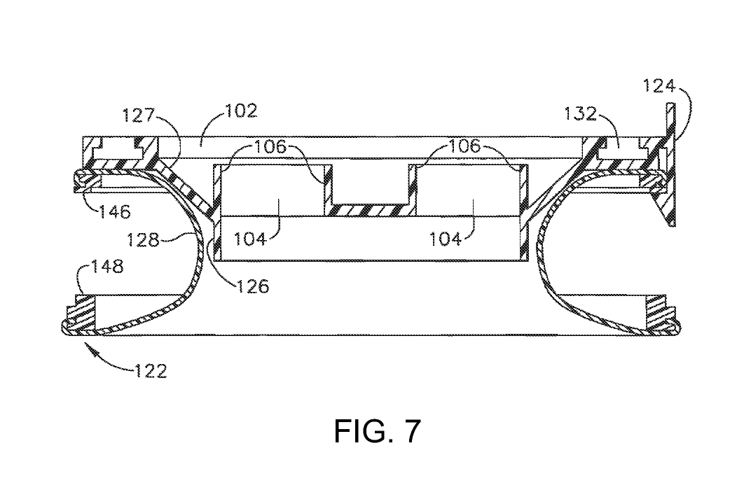

A first aspect of a multi-port insert, generally designated 100, relates to an insert for use with a laparoscopic access device 122. Referring now to the figures, FIG. 2 and FIG. 3 depict one embodiment of the multi-port insert 100. The multi-port insert 100 includes a base 102 having two or more ports or apertures 104 that provide for the insertion of surgical instruments. The multi-port insert 100 may be used with a laparoscopic access device 122 (FIG. 7) such as a Lap Disc Hand Access Device model #LD111, commercially available from Ethicon Endo-Surgery, Inc. of Cincinnati, Ohio. The multi-port insert provides for the insertion of one or more surgical instruments through the laparoscopic access device 122, while preventing insufflation gases from escaping from the body cavity.

As illustrated in FIG. 2 and FIG. 3, the base 102 may include four separate apertures 104 spaced evenly around the center of the base 102 with each aperture 104 having a raised lip or rim 106. This configuration allows surgical tools, such as gripping devices to be inserted through two apertures 104. The gripping devices may be used to manipulate or lift a portion of the bowel to provide the surgeon with access to the either the bowel tissue being manipulated or the underlying tissue. An endoscope including a camera and light may be inserted through a third aperture 104 to provide the surgeon with the ability to view the interior of the body cavity. An additional surgical instrument, such as a needle, scissors, an ultrasonic transducer or any other surgical instrument, may be inserted through the fourth aperture 104. Although FIG. 2 and FIG. 3 illustrate a base 102 including four apertures 104, alternate numbers and configurations of apertures may be used. In addition, a base 102 may include apertures 104 of varying sizes able to provide for the insertion of differently sized surgical instruments. In one embodiment the apertures may be sized to provide for instruments between five and twelve millimeters in diameter. The base 102 may also include an reference indicator 180 that may be used by the surgeon as a reference point during laparoscopic procedures.

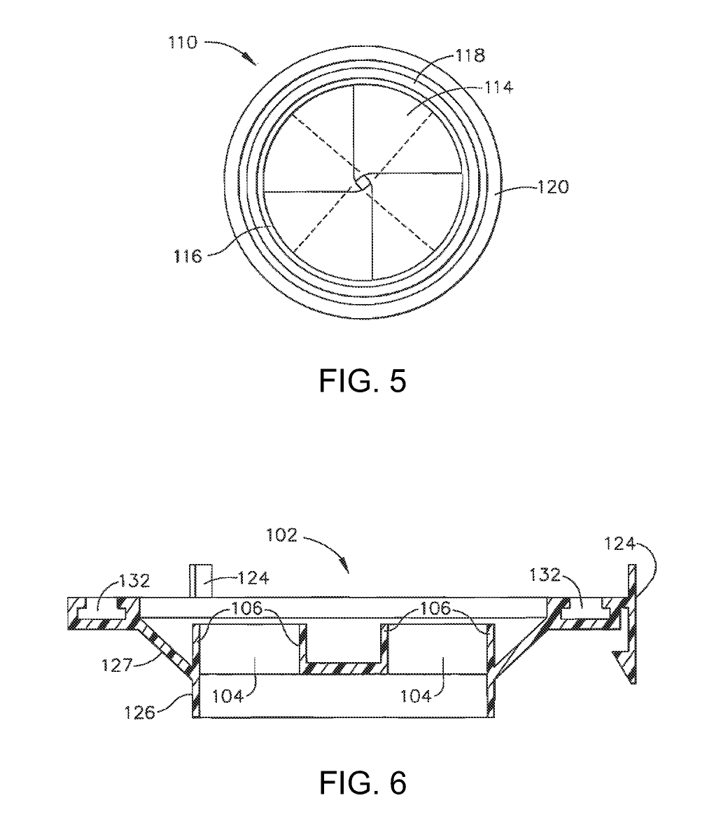

Referring now to FIG. 4 and FIG. 5, each port or aperture 104 in the base 102 includes its own seal assembly 108 to provide a seal and prevent the escape of insufflation gases. There are many possible types of seals which may be utilized in the seal assembly 108. In one embodiment, each seal assembly 108 includes an iris seal 110 and a duck bill valve 112 such as the seal assembly described in U.S. Patent Application Publication No. 2004/0230161 (Ser. No. 10/815,356; filed Mar. 31, 2004) to Zeiner, the entire disclosure of which are hereby incorporated herein by reference. Each iris seal 110 may include a plurality of layered elastic members 114 having a semi-circular profile disposed between two rigid seal rings 116. The elastic members 114 may form a conical-shaped seal such that when a surgical instrument is inserted from the top side thereof, the elastic members 114 are displaced downwardly and radially outwardly and form a seal around the surgical instrument. Each seal assembly 108 may also include a zero-closure valve such as a duckbill valve 112 to prevent the seal assembly 108 from leaking when there is no surgical instrument inserted through the seal assembly 108. The duckbill valve 112 may include two overlapping flaps 113. Pressure from below the duckbill valve 112 pushes the flaps 113 together, maintaining the seal. Pressure from above the duckbill valve 112 pushes the flaps 113 apart, allowing a surgical instrument to pass through.

In one embodiment, each seal assembly 108 is flexibly attached to the base 102 using a floatation system such as bellows 118 located around the periphery of each seal assembly 108. The bellows 118 may be made from a flexible, elastic material and allow the seal assembly 108 to move laterally and pivot within the aperture 104. The movement of the seal assembly 108 allows surgical instruments to be inserted through the apertures 104 at an angle rather than along the axis of the aperture 104. The bellows 118 may be attached to the lip of the aperture 104 by a retaining ring 120 that frictionally fits over each rim 106. The force required to deflect the bellows 118 is much less than the pressure exerted by surgical instrument on the elastic members 114 while the surgical instrument is inserted in the seal assembly 108. This allows the floatation system to deflect within each aperture 104 while the elastic members 114 maintain a sealing condition with the instrument.

The multi-port insert 100 may be attached to a laparoscopic access device 122 as shown in FIG. 7. The laparoscopic access device 122 may include a generally coaxially aligned upper ring 146 and lower ring 148 and a membrane 128 coupled to and extending generally axially between the upper ring 146 and lower ring 148. The membrane 128 has a central opening of variable size. For example, in one embodiment the upper ring 146 and lower ring 148 are rotatable in opposite directions relative to one another to change the size of the opening. The base 102 of the multi-port insert 100 may be attached to the laparoscopic access device 122 by a simple latch mechanism 124 to allow the multi-port insert 100 to be attached to currently available laparoscopic access devices 122. Alternatively, one or more C-clamps or other clamping devices or structures may be used to attach the multi-port insert 100 to a laparoscopic access device 122. In addition, the multi-port insert 100 may be attached to a laparoscopic access device 122 using a threadable surface on the multi-port insert 100 and a corresponding mating threadable surface on the laparoscopic access device 122.

Once attached to the laparoscopic device, the base 102 of the multi-port insert 100 may form a seal with the laparoscopic access device 122 to prevent the escape of insufflation gas. As shown in FIG. 6, the base 102 may include a collar 126 that may be inserted into the laparoscopic access device 122. As illustrated in FIG. 1-7, the collar 126 extends into the laparoscopic access device 122 and forms a seal with the membrane. In addition, the collar 126 may also protect the membrane 128 of the laparoscopic access device from any surgical instruments inserted through the apertures 104. As depicted in FIG. 6, the collar 126 may include a generally tapered portion 127. The tapered portion 127 allows the apertures 104 to be seated within the laparoscopic access device 122. Lowering the apertures 104 lowers the pivot points of the surgical instruments and increases the range of motion of the surgical instruments inserted through the apertures 104. In an alternate embodiment, the collar 126 does not include a tapered portion and may be generally cylindrically shaped.

In another embodiment, the base 102 of the multi-port insert may be inserted through the opening in the membrane 128 of the laparoscopic access device 122 and attached to the lower ring 148 of the laparoscopic access device 122. This configuration would provide a greater range of motion within the body cavity for the surgical instruments by lowering the pivot points for the instruments below the surface of the skin.

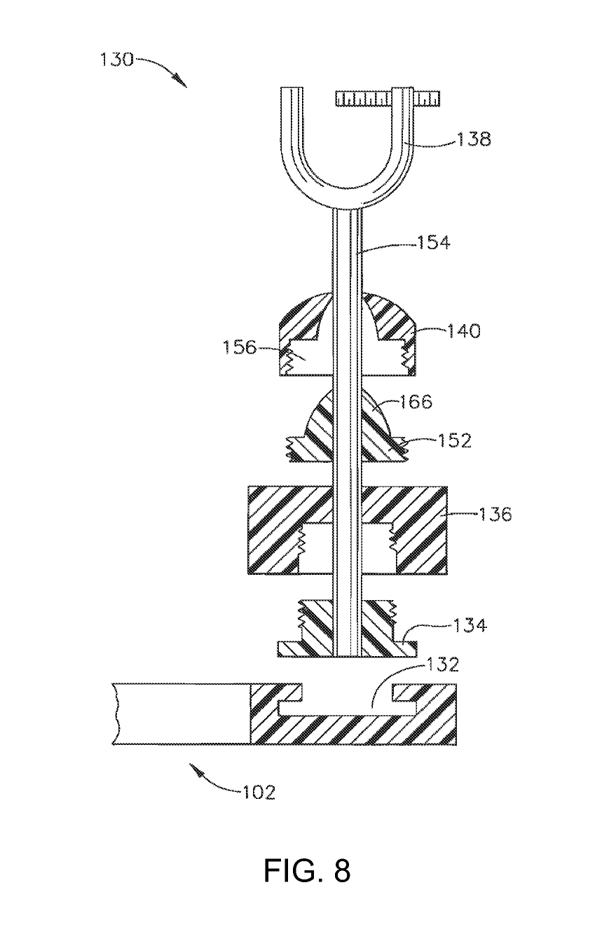

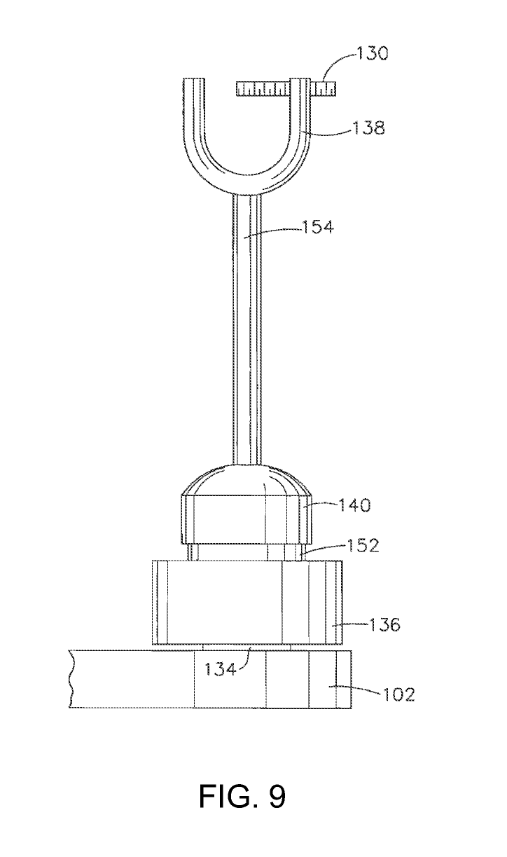

The multi-port insert 100 may also include one or more instrument supports 130 that are attached to the base 102 to fix the position of one or more surgical instruments inserted through the multi-port insert 100. FIG. 8 illustrates a first embodiment of a surgical instrument support 130 extending generally axially from the base 102. The surgical instrument support 130 may include a gripping portion 138, a stem 154 and an instrument support base 134. The gripping portion 138 may be used to hold one or more surgical instruments and may include a C-clamp or any other device suitable for holding a surgical instrument. The stem 154 connects the gripping portion 138 and the instrument support base 134. In one embodiment, the stem 154 may be composed of a malleable substance, such as copper wire, to allow the surgical instrument support 130 to be positioned to hold a surgical instrument.

The instrument support base 134 attaches the instrument support 130 to the multi-port insert 100. The base 134 may be inserted into a track 132 that extends around the periphery of the multi-port insert 100. The track 132 may include an opening 178 to allow an instrument support base 134 to be inserted into the track 132. The instrument support 130 may be positioned along the track 132 around the circumference of the multi-port insert 100.

The instrument support 130 includes a positional lock 136 for fixing the position of the instrument support base 134 with respect to the multi-port insert 100. The stem 154 may be inserted through an aperture in the positional lock 136. The positional lock 136 may be threadably connected to the instrument support base 134, such that when the positional lock 136 is rotated in a first direction the instrument support base 134 is drawn upward away from the base 102. Frictional forces between the track 132, the instrument support base 134 and the positional lock 136 secure the instrument support base 134 relative to the base 102 of the multi-port insert 100. In an alternative embodiment, a clamp may be used to secure the instrument support 130 to the base 102.

The instrument support 130 may also include an extension control 152 and an extension lock 140. The extension control 152 includes a generally conical portion 166 and an aperture shaped to receive the stem 154. The conical portion 166 of the extension control 152 includes one or more slits (not shown). The extension lock 140 includes an aperture shaped to receive the stem 154 and a generally conical shaped opening 156. The extension lock 140 may be threadably connected to the extension control 152 such that the extension lock 140 may be drawn downward over the extension control 152. The pressure exerted by the extension lock 140 on the extension control 152 pushes the conical portion 166 of the extension control 152 down and inward, exerting pressure against the stem 154, preventing the stem 154 from sliding through the apertures in the extension control 152 and thereby locking the stem 154 in place. The surface of the stem 154 may be rough, textured or covered with a coating to increase friction between the stem and the extension control 152 and facilitate locking the stem 154 in place.

The multi-port insert may be utilized during laparoscopic procedures to provide the surgeon with the ability to insert multiple surgical instruments into the body cavity of the patient without substantial loss of insufflation gases and without requiring multiple additional incisions. In one embodiment the lower ring of the laparoscopic access device 122 may be inserted into the body of a patient through an incision in the abdomen of the patient. During laparoscopic surgery, the surgeon may elect to attach a multi-port insert 100 to the upper ring 148 of the laparoscopic access device 122 using the latch mechanisms 124, clamps or the like. Once attached, the multi-port insert 100 forms a seal with the laparoscopic access device 122. The seal between the multi-port insert 100 and the laparoscopic access device 122 and the seal assemblies 108 prevent excessive amounts of the insufflation gases from escaping the body cavity. The surgeon may insert a surgical instrument through any or all of the apertures 104. This allows the surgeon to insert multiple surgical instruments into the body cavity patient at the same time. The seal assemblies 108 automatically reseal upon removal of the surgical instruments allowing the surgeon to insert and remove multiple surgical instruments during surgery.

The multi-port insert 100 may also include one or more instrument supports 130 designed to hold surgical instruments inserted through the multi-port insert. In one embodiment the instrument supports 130 attach to the track 132 in the base 102. The instrument supports 130 may be positioned at an appropriate location on the base 102 and locked into place using the positional lock 136. The surgeon may control the distance the instrument support 130 extends from the base 102 using the extension control 152 and extension lock 140. The instrument support 130 may be attached to a surgical instrument using the gripping portion 138. The surgeon may reposition and readjust the instrument support 130 at any time. At any time during the procedure the surgeon may elect to disconnect the multi-port insert 100 from the laparoscopic access device 122. Various other devices are disclosed in U.S. patent application Ser. No. 11/398,985, entitled MULTI-PORT LAPAROSCOPIC ACCESS DEVICE, which was filed on Apr. 5, 2006, the entire disclosure of which is incorporated by reference herein.

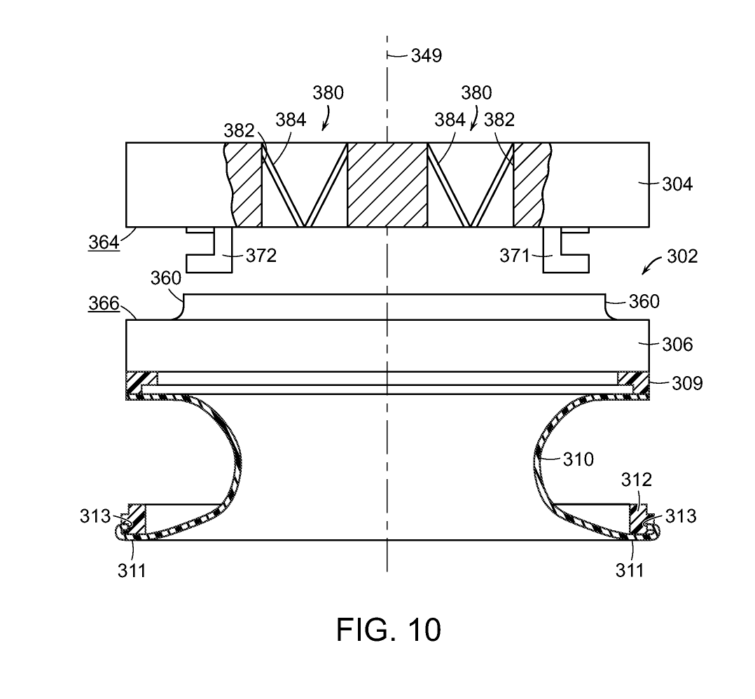

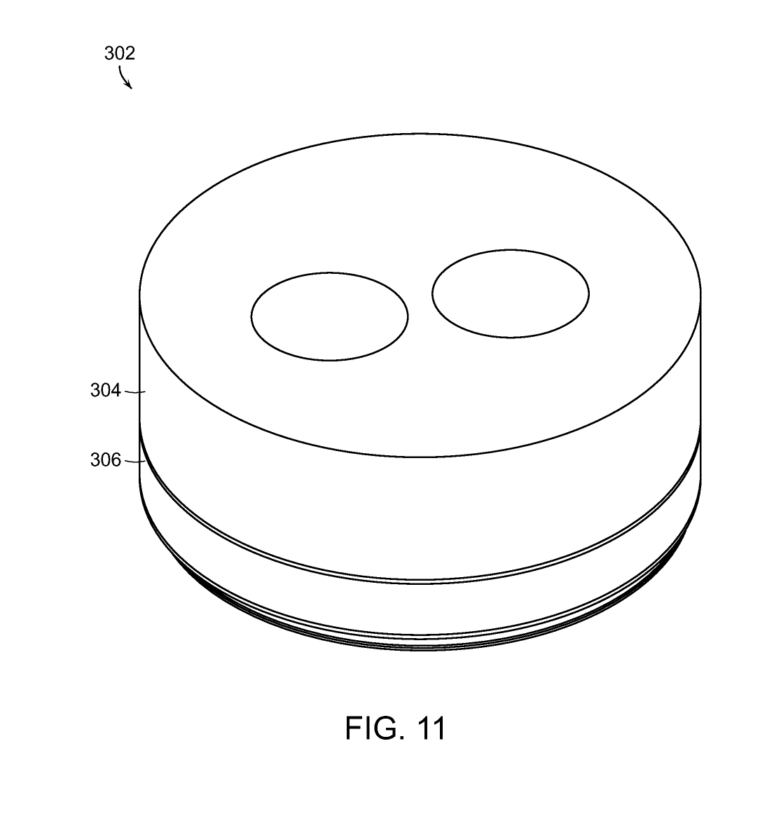

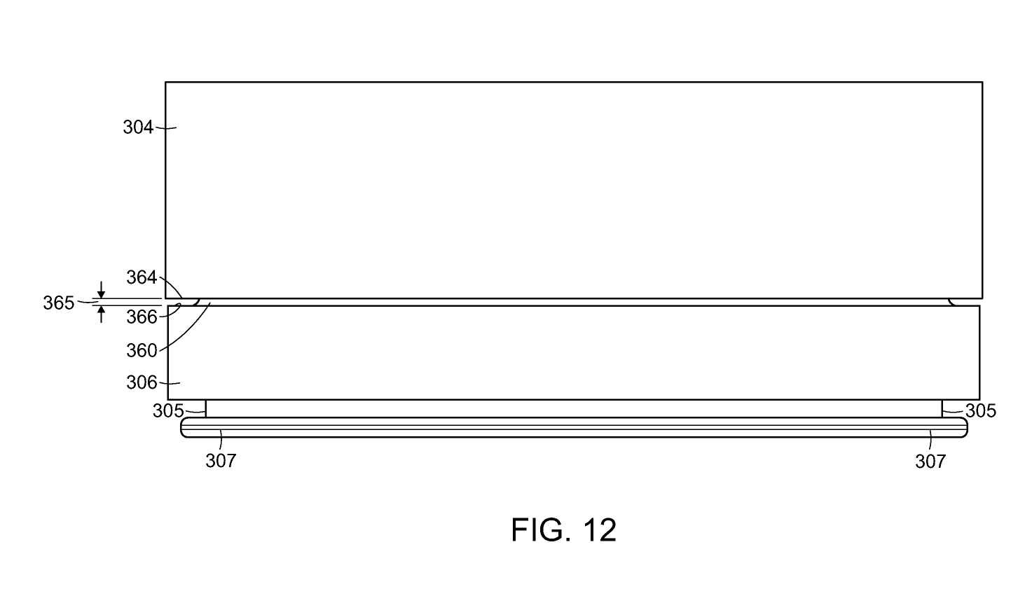

Similar to access device 202, referring now to FIG. 10, an access device 302 can be positioned within an incision in order to access a surgical site within the patient. The access device 302 can comprise a lower ring, or base member, 306 which can be configured to be positioned against the patient's body and, in addition, an insertable ring, or insertable member, 312 which can be configured to be inserted through the incision. In various embodiments, the rings 306 and/or 312 can be annular, or at least substantially annular, for example, although they can comprise any suitable configuration. The access device 302 can further comprise a flexible sleeve 310 mounted to the lower ring 306 and the insertable ring 312. In various embodiments, a proximal end 309 of sleeve 310 can be attached to lower ring 306 within a groove 305 (FIG. 12), for example. More particularly, in at least one embodiment, the proximal end 309 can be resiliently expanded and inserted over ridge 307 and then positioned within the groove 305. In certain embodiments, a retaining ring can be positioned around the proximal end 309, such as within groove 305, for example, in order to secure the proximal end 309 to the lower ring 306. In various embodiments, the groove 305, the ridge 307, and/or the retaining ring can extend around at least a portion or, or the entirety of, the lower ring 306. Similarly, referring again to FIG. 10, a distal end 311 of the flexible sleeve 310 can be attached to insertable ring 312 within a groove 313, for example. In various embodiments, further to the above, the groove 313 can extend around at least a portion of, or the entirety of, insertable ring 312 wherein a retaining ring can be utilized to hold the distal end 311 in groove 313, for example.

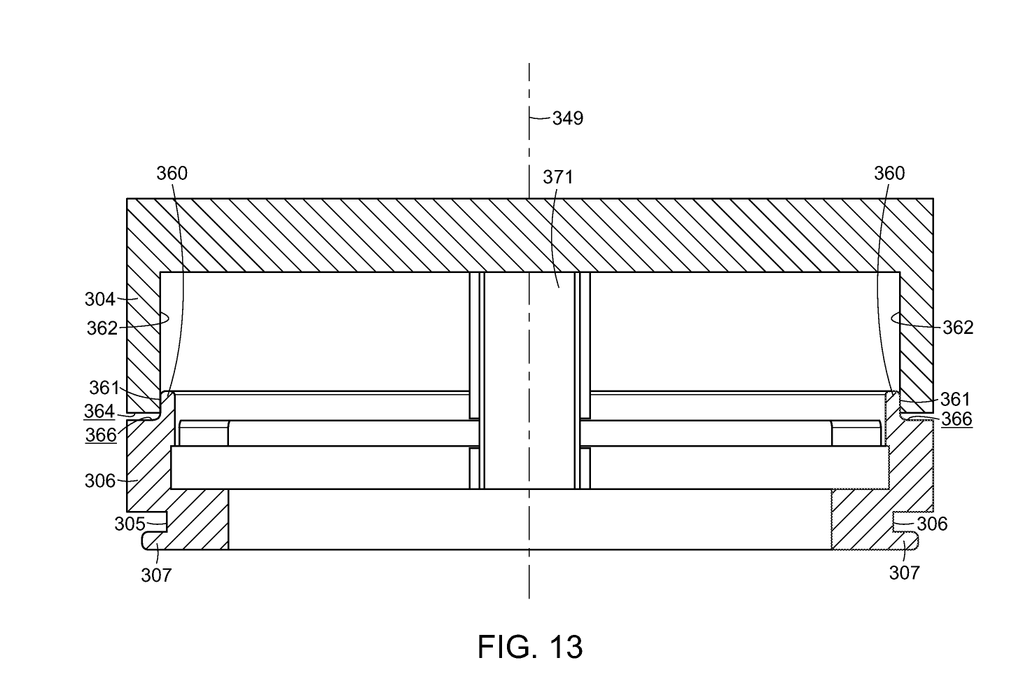

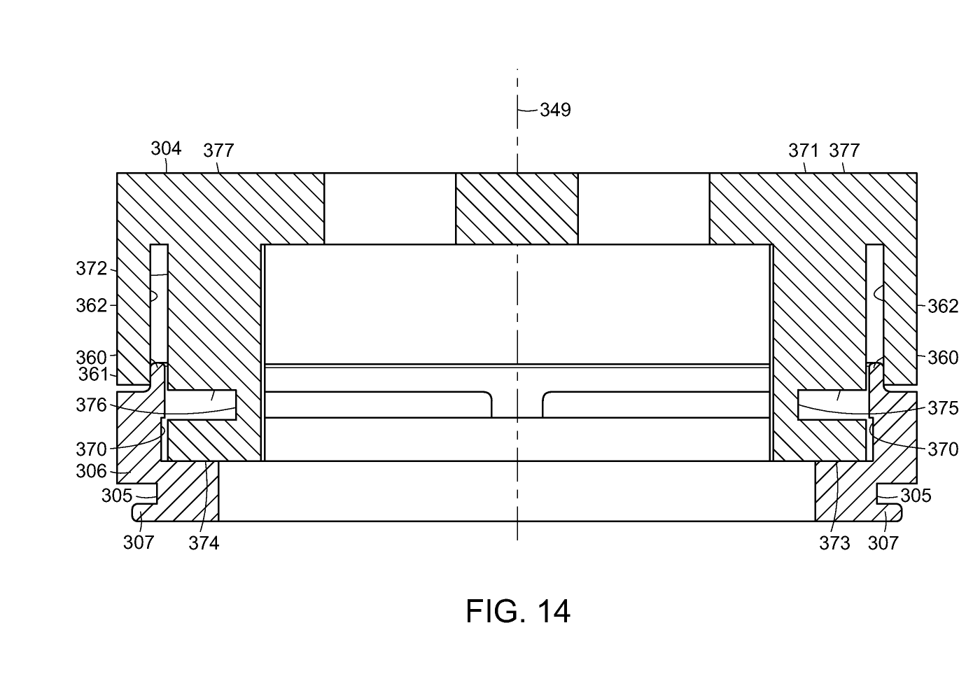

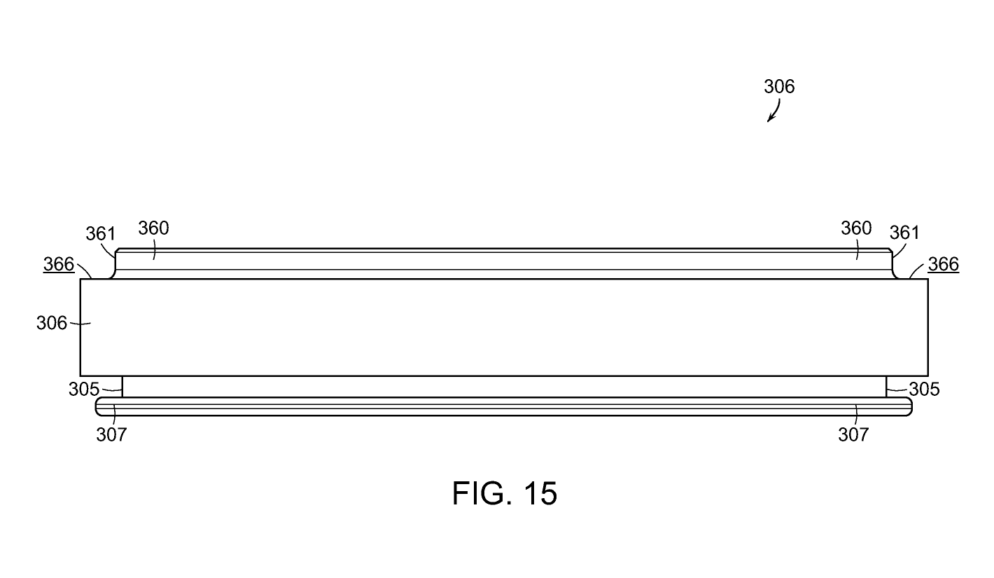

In various embodiments, further to the above, an upper ring, or cap, such as upper ring 304, for example, can be positioned against the lower ring 306. In at least one embodiment, the lower ring 306 can comprise a central portion 360 about which the upper ring 304 can be rotated. More particularly, in at least one embodiment, the central portion 360 can comprise an annular, or at least substantially annular, ridge or wall extending upwardly which can be configured to be closely received within, referring to FIGS. 13 and 14, an inner aperture, or slot, 362 in upper ring 304. In at least one embodiment, the outer perimeter of the central portion 360 can be sized and configured such that there is abutting contact with a sidewall of the aperture 362 and such that said upper ring 304 rests upon said central portion wall 360, for example. In at least one such embodiment, the outer perimeter of the central portion 360 can comprise an curved profile 361 against which the upper ring 304 can be positioned. In various embodiments, the curved profile 361 can be at least one of arcuate, convex, and/or concave, for example. In certain embodiments, profile 361 may be linear and can define a wall 360 which is thicker distally than it is proximally, for example. Owing to the above, the central portion 360 can define an axis 349 about which the upper ring 304 can be rotated. In various embodiments, referring again to FIG. 13, the sidewall of aperture 362 can be in contact with the central portion wall 360 such that the bottom surface 364 of upper ring 304 is not in contact with the top surface 366 of bottom ring 306. In at least one such embodiment, referring now to FIG. 12, the surfaces 364 and 366 can define a gap 365 therebetween. In various alternative embodiments, the surfaces 364 and 366 can be in contact with one another. In any event, as described above, the upper ring 304 and the bottom ring 306 can be rotated relative to one another.

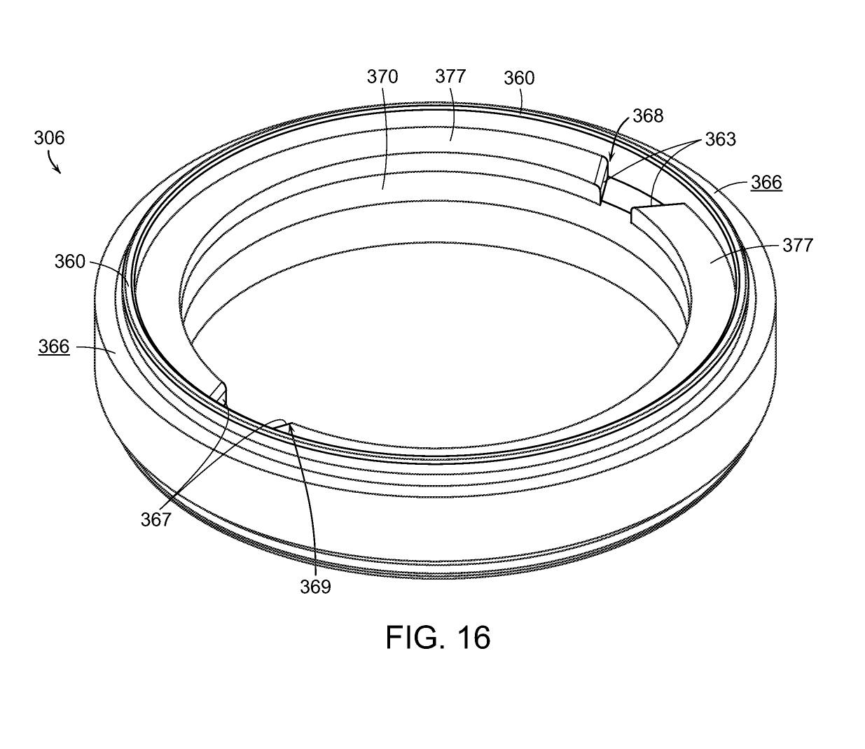

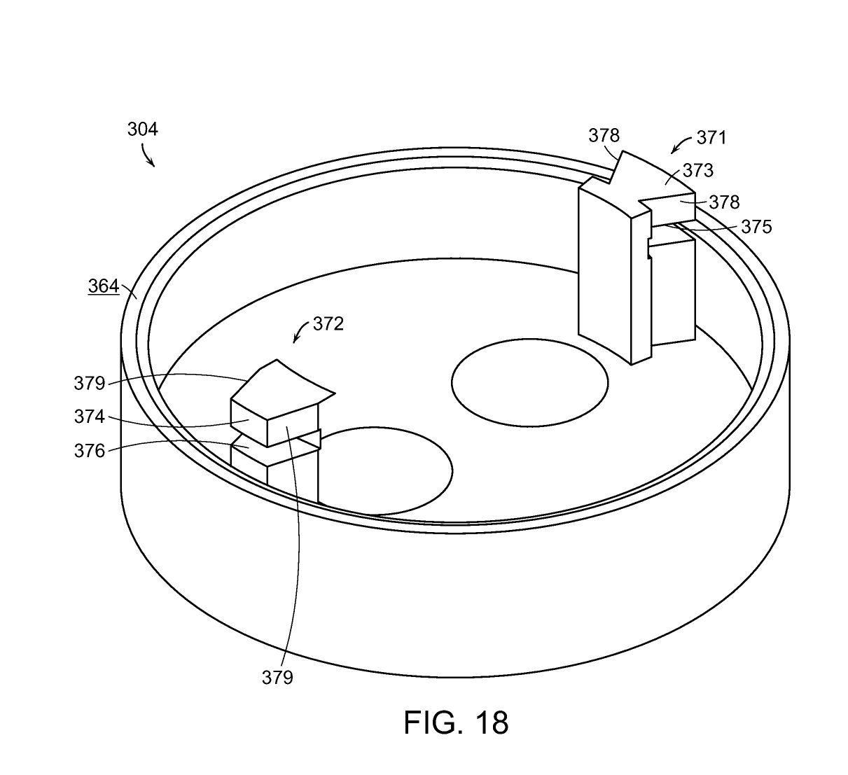

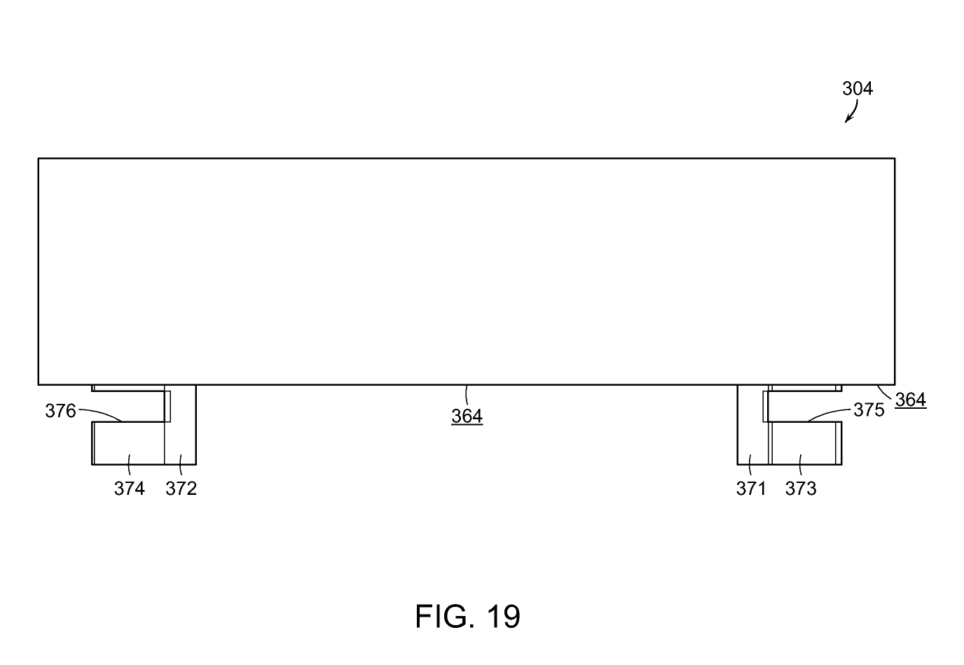

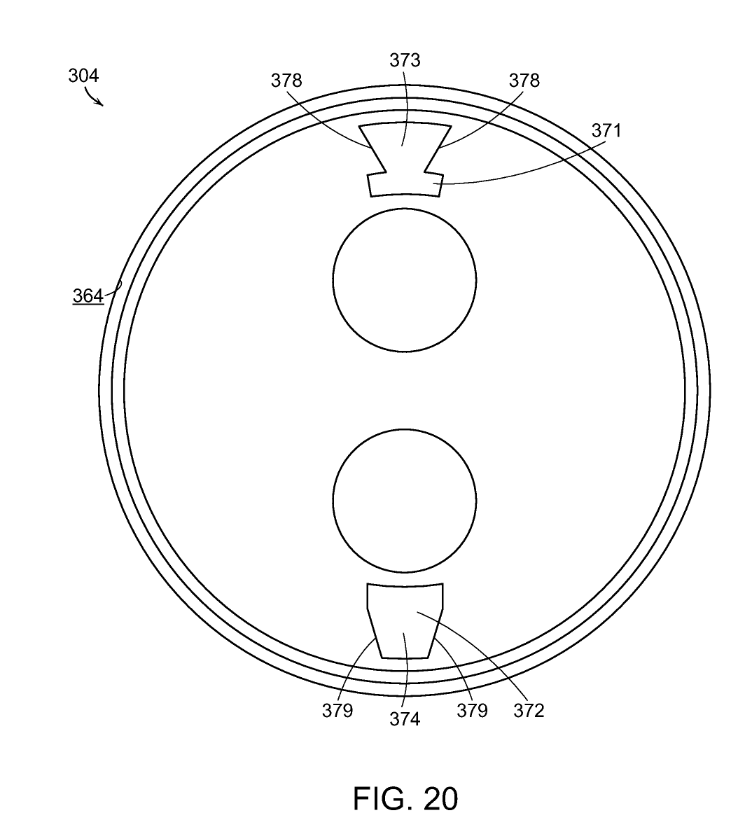

In various embodiments, further to the above, the upper ring 304 can be removably attachable to the lower ring 306. In at least one embodiment, referring now to FIG. 14, the bottom ring 306 can comprise an annular, or at least substantially, inner track, or keyway, 370 extending around the inner perimeter thereof. Generally referring now to FIGS. 18 and 19, the upper ring 304 can comprise one or more retention members, such as retention members 371 and 372, for example, which can be configured to be inserted into inner track 370 in order to retain the upper ring 304 to the lower ring 306. In at least one embodiment, referring to FIGS. 16 and 18, the retention member 371 can comprise a foot, or key, 373 and a slot 375, wherein the foot 373 can be configured to be inserted into inner track 370 via an access slot 368 and, in addition, the retention member 372 can comprise a foot, or key, 374 and a slot 376, wherein the foot 374 can be configured to be inserted into inner track 370 via an access slot 369. Once feet 373, 374 are positioned within access slots 368, 369, respectively, the upper ring 308 can be rotated relative to the lower ring 306 in order to lock the upper ring 304 to the lower ring 306. More particularly, the upper ring 304 can be rotated such that feet 373 and 374 are rotated underneath upper lip 377, which defines the upper wall of inner track 370, and such that the upper lip 377 has been received within slots 375, 376 of retention members 371, 372, respectively. As a result of the above, the upper ring 304 cannot be lifted upwardly away from the lower ring 306 unless the upper ring 304 is retuned to the unique position in which the retention member 371 is aligned with access slot 368 and the retention member 372 is aligned with the access slot 369, as described in greater detail below.

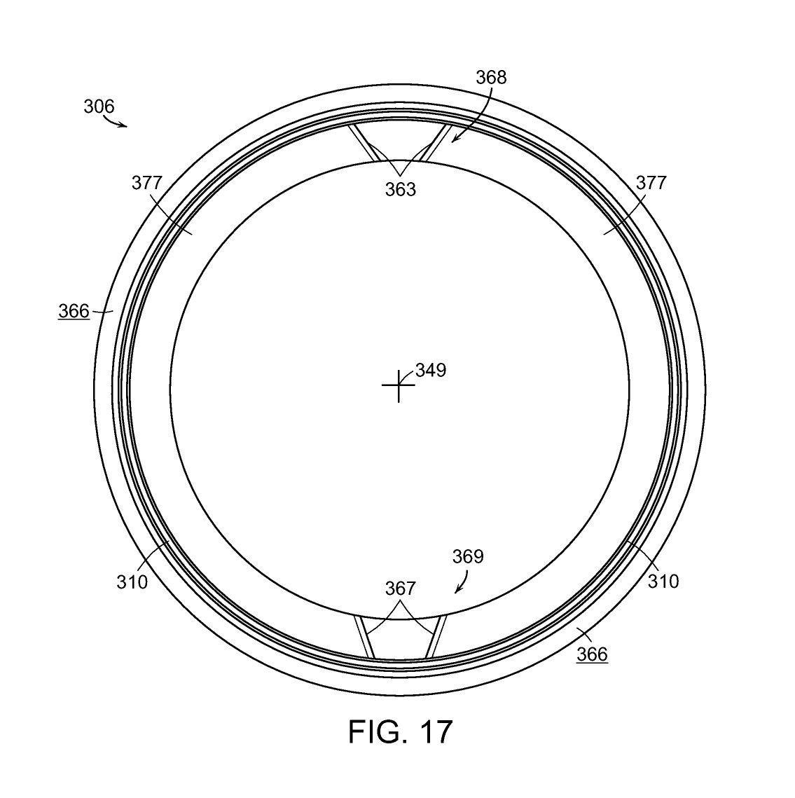

Once the upper ring 304 has been rotated relative to the bottom ring 306 and the feet 373 and 374 have been slid under lip 377, the upper ring 304 can be rotated an entire revolution about axis 349 before the feet 373, 374 become aligned with access slots 368, 369, respectively, once again. In at least one such embodiment, the upper ring 304 cannot be removed from the bottom ring 306 until the foot 373 has become aligned with access slot 368 and the foot 374 has become aligned with the access slot 369. More particularly, in at least one embodiment, the access slots 368, 369 and the feet 373, 374 can be configured such that foot 373 can only be removed from inner track 370 via access slot 368 and such that foot 374 can only be removed from inner track 370 via access slot 369. Referring to FIG. 17, access slot 368 can comprise non-parallel sidewalls 363 which are oriented along axes which can converge toward the center axis 349. Correspondingly, referring to FIG. 18, the foot 373 of retention member 371 can comprise non-parallel sidewalls 378 which can also be oriented along axes which can converge toward the center axis 349, wherein the sidewalls 378 can be parallel, or at least substantially parallel, to the sidewalls 363 of access slot 368. Referring again to FIG. 17, access slot 369 can comprise non-parallel sidewalls 367 which are oriented along axes which can converge away from the center axis 349. Correspondingly, referring to FIG. 18, the foot 374 of retention member 372 can comprise non-parallel sidewalls 379 which can also be oriented along axes which can converge away from the center axis 349, wherein the sidewalls 379 can be parallel, or at least substantially parallel, to the sidewalls 367 of access slot 369.

In various embodiments, as described above, the upper ring 304 can be assembled to the lower ring 306 and then rotated relative thereto. In at least one embodiment, similar to the above, the upper ring 304 can comprise one or more apertures, or ports, through which a surgical instrument can be inserted. Referring now to FIG. 10, the upper ring 304 can comprise ports 380 which, in at least one embodiment, can each be defined by a distally, or downwardly, depending sidewall 382. In various embodiments, each port 380 can comprise one or more seals, such as seals 384, for example, which can be configured to flex toward sidewalls 382 when a surgical instrument is inserted therethrough and, as a result, maintain sealing contact with the surgical instrument. In use, the upper ring 304 can be rotated relative to the bottom ring 306 in order to re-position the ports 380 and the surgical instruments extending therethrough. In various circumstances, owing to the co-operative arrangement of feet 373, 374 and lip 377, as described above, the upper ring 304 can remain retained to the lower ring 306. More particularly, as also described above, the feet 373, 374 and the access slots 368, 369 can be configured such that the upper ring 304 cannot be detached from the lower ring 306 except for one specific orientation, or alignment, between the upper ring 304 and the lower ring 306. In any event, in at least one embodiment, a seal can be positioned intermediate the upper ring 304 and the lower ring 306, such as between surfaces 364 and 366, for example, wherein the seal can be configured to prevent, or at least inhibit, insufflation gasses from flowing thereby. In at least one such embodiment, the seal can increase the force required to overcome the friction force between the upper ring 304 and the lower ring 306 in order to move the upper ring 304 relative to the lower ring 306.

The embodiments of the devices described herein may be introduced inside a patient using minimally invasive or open surgical techniques. In some instances it may be advantageous to introduce the devices inside the patient using a combination of minimally invasive and open surgical techniques. Minimally invasive techniques may provide more accurate and effective access to the treatment region for diagnostic and treatment procedures. To reach internal treatment regions within the patient, the devices described herein may be inserted through natural openings of the body such as the mouth, anus, and/or vagina, for example. Minimally invasive procedures performed by the introduction of various medical devices into the patient through a natural opening of the patient are known in the art as NOTES™ procedures. Some portions of the devices may be introduced to the tissue treatment region percutaneously or through small—keyhole—incisions.

Endoscopic minimally invasive surgical and diagnostic medical procedures are used to evaluate and treat internal organs by inserting a small tube into the body. The endoscope may have a rigid or a flexible tube. A flexible endoscope may be introduced either through a natural body opening (e.g., mouth, anus, and/or vagina) or via a trocar through a relatively small—keyhole—incision incisions (usually 0.5-1.5 cm). The endoscope can be used to observe surface conditions of internal organs, including abnormal or diseased tissue such as lesions and other surface conditions and capture images for visual inspection and photography. The endoscope may be adapted and configured with working channels for introducing medical instruments to the treatment region for taking biopsies, retrieving foreign objects, and/or performing surgical procedures.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. Other sterilization techniques can be done by any number of ways known to those skilled in the art including beta or gamma radiation, ethylene oxide, and/or steam.

Although the various embodiments of the devices have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A surgical access device for providing surgical access through an incision in a body wall of a patient to the inside of a body cavity of the patient, the surgical access device having a center axis and comprising:
an assembly comprising a proximal end and a distal end, said assembly further comprising an opening around said center axis, said assembly comprising:
a ring comprising a track slot and an access slot; and
a sleeve coupled to and extending generally axially from said ring and positionable in the incision, said sleeve defining a passageway through the body wall between said opening in said assembly and the body cavity; and
a subassembly releasably positionable at said proximal end of said assembly, wherein said subassembly comprises a key configured to be inserted through said access slot into said track slot, wherein said key is configured to slide within said track slot when said subassembly is rotated about said center axis, and wherein said rotation of said subassembly about said axis can comprise a full rotation about said axis.

2. The surgical access device of claim 1, wherein said key comprises a first key and said access slot comprises a first access slot, wherein said ring comprises a second access slot, wherein said subassembly further comprises a second key configured to be inserted through said second access slot into said track slot, and wherein said second key is configured to slide within said track slot when said subassembly is rotated about said center axis.

3. The surgical access device of claim 2, wherein said first key is configured such that it cannot be inserted through said second access slot, and wherein said second key is configured such that it cannot be inserted through said first access slot.

4. The surgical access device of claim 1, wherein said subassembly comprises a cap, a cap aperture extending through said cap, and a seal operably coupled with said cap aperture, and wherein said seal is configured to at least partially sealingly engage an instrument inserted through said cap aperture.

5. The surgical access device of claim 1, wherein said ring comprises an annular wall extending proximally therefrom, wherein said subassembly is configured to rest upon said annular wall, and wherein said annular wall defines said central axis.

6. The surgical access device of claim 5, wherein said annular wall comprises a curved outer surface, and wherein said subassembly is configured to contact said curved outer surface.

7. A surgical access device for providing surgical access through an incision in a body wall of a patient to the inside of a body cavity of the patient, the surgical access device comprising:
an assembly comprising:
a proximal end;
a distal end,
an opening;
a base member comprising an annular track slot and an access slot; and
a flexible sleeve coupled to and extending generally axially from said base member and positionable in the incision, said flexible sleeve defining a passageway through the body wall between said opening in said assembly and the body cavity; and
a cap releasably positionable at said proximal end of said assembly, wherein said cap comprises a retention member configured to be inserted through said access slot into said track slot, wherein said retention member is configured to slide within said track slot when said cap is rotated relative to said base member, and wherein said rotation of said cap relative to said base member can comprise a full rotation through which said cap cannot be removed from said base member unless said cap and said base member are aligned in a unique relative position.

8. The surgical access device of claim 7, wherein said retention member comprises a first retention member and said access slot comprises a first access slot, wherein said base member comprises a second access slot, wherein said cap further comprises a second retention member configured to be inserted through said second access slot into said track slot, and wherein said second retention member is configured to slide within said track slot when said cap is rotated relative to said base member.

9. The surgical access device of claim 8, wherein said first retention member is configured such that it cannot be inserted through said second access slot, and wherein said second retention member is configured such that it cannot be inserted through said first access slot.

10. The surgical access device of claim 7, wherein said cap further comprises a cap aperture extending through said cap and a seal operably coupled with said cap aperture, and wherein said seal is configured to at least partially sealingly engage an instrument inserted through said cap aperture.

11. The surgical access device of claim 7, wherein said base member comprises an annular wall extending proximally therefrom, wherein said cap is configured to rest upon said annular wall, and wherein said annular wall defines a central axis about which said cap can be rotated relative to said base member.

12. The surgical access device of claim 11, wherein said annular wall comprises a curved outer surface, and wherein said cap is configured to contact said curved outer surface.

13. A surgical access device for providing surgical access through an incision in a body wall of a patient to the inside of a body cavity of the patient, the surgical access device comprising:
an assembly, comprising:
a proximal end;
a distal end;
an opening;
a base member comprising:
an annular track slot;
a first access slot; and
a second access slot;
a flexible sleeve coupled to and extending generally axially from said base member and positionable in the incision, said flexible sleeve defining a passageway through the body wall between said opening in said assembly and the body cavity; and
a cap releasably positionable at said proximal end of said assembly, wherein said cap comprises:
a first retention member configured to be inserted through said first access slot into said track slot; and
a second retention member configured to be inserted through said second access slot into said track slot, wherein said first retention member and said second retention member are configured to slide within said track slot when said cap is rotated relative to said base member, and wherein said rotation of said cap about said axis can comprise a full rotation through which said cap cannot be removed from said base member except when said first retention member is aligned with said first access slot and said second retention member is aligned with said second access slot.

14. The surgical access device of claim 13, wherein said first retention member is configured such that it cannot be inserted through said second access slot, and wherein said second retention member is configured such that it cannot be inserted through said first access slot.

15. The surgical access device of claim 13, wherein said cap further comprises a cap aperture extending through said cap and a seal operably coupled with said cap aperture, and wherein said seal is configured to at least partially sealingly engage an instrument inserted through said cap aperture.

16. The surgical access device of claim 13, wherein said base member comprises an annular wall extending proximally therefrom, wherein said cap is configured to rest upon said annular wall, and wherein said annular wall defines a central axis about which said cap can be rotated relative to said base member.

17. The surgical access device of claim 16, wherein said annular wall comprises a curved outer surface, and wherein said cap is configured to contact said curved outer surface.
